(19) United States
(12) Patent Application Publication
(10) Pub. No.: US 2015/0231117 A1
(43) Pub. Date: Aug. 20, 2015

(54) ARRY-520 FOR USE IN TREATING CANCER IN A PATIENT WITH LOW AAG

(71) Applicant: Array BioPharma Inc., Boulder, CO (US)

(72) Inventors: Karin Brown, Boulder, CO (US); Ronald B. Franklin, Boulder, CO (US); Gary P. Hingorani, Boulder, CO (US); Kevin S. Litwiler, Boulder, CO (US); Brian J. Tunquist, Boulder, CO (US); Duncan H. Walker, Boulder, CO (US)

(73) Assignee: Array BioPharma Inc., Boulder, CO (US)

(21) Appl. No.: 14/421,766

(22) PCT Filed: Aug. 13, 2013
(86) PCT No.: PCT/US2013/054807

§ 371 Date: Feb. 13, 2015

Related U.S. Application Data
(60) Provisional application No. 61/829,779, filed on May 31, 2013.
(60) Provisional application No. 61/734,149, filed on Dec. 6, 2012.
(60) Provisional application No. 61/682,682, filed on Aug. 13, 2012.

(51) Int. Cl. A61K 31/433; A61K 38/05; G01N 33/574; A61K 31/454; A61K 38/19; A61K 31/573; A61K 38/07

(52) U.S. Cl. CPC A61K 31/433; A61K 31/573; A61K 38/05; A61K 38/07; A61K 31/454; A61K 38/193; G01N 33/57488; G01N 2333/4728

(57) ABSTRACT

The compound ARRY-520 for use for treating cancer in patients with low [AAG] is provided.

### RELATED APPLICATIONS

The present application claims priority of U.S. Provisional Patent Application No. 61/682,682, filed Aug. 13, 2012, U.S. Provisional Patent Application No. 61/734,149, filed Jan. 3, 2013, and U.S. Provisional Patent Application No. 61/829,779, filed May 31, 2013, and which applications are incorporated by reference herein in their entireties.

### BACKGROUND OF THE INVENTION

1. Field of the Invention

The present invention relates to ARRY-520 and treating cancer patients having low [AAG].

2. Description of the State of the Art

(S)-2-(3-Aminopropyl)-5-(2,5-difluorophenyl)-N-methoxy-N-methyl-2-phenyl-1,3,4-thiadiazole-3(2H)-carboxamide, also known as “ARRY-520”, which has the structure:

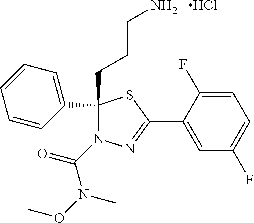 [SMILES: CON(C)C(=O)N1N=C(c2cc(F)ccc2F)S[C@@]1(CCCN)c1ccccc1 ; Cl]

is a kinesin spindle protein (“KSP”) inhibitor (see U.S. Pat. No. 7,449,486, US 2010/0099697 and WO 2010/045624, the contents of which are herein incorporated by reference in their entirety). KSP inhibition results in mitotic arrest of proliferating cells and subsequent cell death. ARRY-520 has shown clinical activity in patients with relapsed and refractory multiple myeloma (“MM”). Despite intravenous (“IV”) administration, ARRY-520 pharmacokinetics (“PK”) is variable among patients.

Serum protein binding to drugs can alter their potency and PK, and possibly impact clinical activity. Human serum albumin (“HSA”) and human a 1-acid glycoprotein (“AAG”) are the most abundant plasma proteins with average physiological concentrations of approximately 40 g/L and 0.6-1.2 g/L, respectively. AAG is an acute-phase serum protein produced by the liver in response to inflammation and infection. Although some extra-hepatic expression has been reported, AAG is predominantly produced in the liver. AAG is sometimes elevated in blood of patients with cancer, including multiple myeloma. AAG plasma levels can vary due to physiological, pathological and genetic factors. The fluctuations in AAG plasma levels can have a direct effect on concentrations of unbound drug, and consequently alter the drugs PK and pharmacodynamics (“PD”). When the concentration of AAG is high it is associated with reduced response and progression free survival (“PFS”) for drugs that tightly bind to AAG (Bruno, Rene, et al. “α-1-Acid Glycoprotein As an Independent Predictor for Treatment Effects and a Prognostic Factor of Survival in Patients with Non-small Cell Lung Cancer Treated with Docetaxel.” Clin. Cancer Res. Vol. 9 (2003): pp 1077-1082). At diagnosis, the concentration of AAG for multiple myeloma patients ranged from 0.4 to 4.1 g/L, with 24% having a high concentration of AAG (Felliniemi Tarja-Terttu, et al. “Immunoreactive Interleukin-6 and Acute Phase Proteins as Prognostic Factors in Multiple Myeloma.” Blood. Vol. 85, No. 3 (Feb. 1, 1995): pp. 765-771). See also, Brown, Karin D., et al. “An Effective Screening Approach to Assess the Impact of α-1 Acid Glycoprotein Binding on the Fraction Unbound of a Drug.” 17^{th} North American Regional International Society for the Study of Xenobiotics Meeting, Atlanta, Ga., Oct. 18, 2011, Abstract #25022, www.arraybiopharma.com/_documents/Publication/PubAttachment479.pdf.

### SUMMARY OF THE INVENTION

Surprisingly, it has been found that patients who have been administered ARRY-520 respond better when those patients have a low [AAG] prior to the administration of ARRY-520.

In one aspect, the present invention relates to ARRY-520 for use in treating cancer in a patient with low [AAG].

In another aspect, ARRY-520 for use in treating cancer in a patient, comprising (a) assaying a biological sample from the patient for the [AAG], (b) determining whether the sample has low [AAG], and (c) administering a therapeutically effective amount of ARRY-520 to the patient if they have low [AAG] is provided.

In another aspect, ARRY-520 for use in treating cancer in a patient, comprising (a) obtaining a biological sample from the patient; (b) assaying the biological sample for the [AAG], (c) determining whether the sample has low [AAG], and (d) administering a therapeutically effective amount of ARRY-520 to the patient if they have low [AAG] is provided.

In another aspect, a method for treating cancer in a cancer patient identified as having low [AAG] comprising a step of treating the patient with ARRY-520, comprising: (a) identifying the patient as having low [AAG] by assaying a biological sample from the patient, and (b) administering ARRY-520 to the patient having low [AAG] is provided.

In another aspect, a method for treating cancer in a cancer patient identified as having low [AAG] comprising a step of treating the patient with ARRY-520, comprising: (a) obtaining a biological sample from the patient; (b) identifying the patient as having low [AAG] by assaying the biological sample from the patient, and (c) administering ARRY-520 to the patient having low [AAG] is provided.

In another aspect, a method of detecting a patient more likely to respond to ARRY-520, comprising obtaining a biological sample from the patient and assaying the sample to determine the [AAG], wherein low [AAG] is indicative of a patient more likely to respond to ARRY-520 is provided.

In another aspect, a method of detecting a patient more likely to respond to ARRY-520, comprising obtaining a biological sample from the patient, assaying the sample to determine the [AAG], and determining whether the patient is more likely to respond to ARRY-520, wherein low [AAG] is indicative of a patient more likely to respond to ARRY-520 is provided.

In another aspect, a method for increasing the likelihood of response in a patient having cancer, comprising: (a) identifying the patient as having low [AAG] by assaying the biological sample from the patient; and (b) administering ARRY-520 to the patient classified as having an increased likelihood of response is provided.

In another aspect, a method for increasing the likelihood of response in a patient having cancer, comprising: (a) obtaining a biological sample from the patient; (b) contacting the sample with an assay to measure the [AAG]; (c) determining whether the sample has low [AAG]; (d) classifying the patient as having an increased likelihood of response if the patient has low [AAG]; and (e) administering ARRY-520 to the patient classified as having an increased likelihood of response is provided.

In another aspect, method for predicting an increased likelihood a patient will respond therapeutically to a method of treating cancer comprising administering ARRY-520, the method comprises: (a) measuring the [AAG] in a biological sample of the patient; (b) determining whether the sample has low [AAG], (c) classifying the patient as having an increased likelihood of responding therapeutically to the method of treating cancer if the sample has low [AAG], and (d) administering ARRY-520 to the patient classified as having an increased likelihood of response is provided.

In another aspect, a method for predicting an increased likelihood a patient will respond therapeutically to a method of treating cancer comprising administering ARRY-520, the method comprises: (a) obtaining a biological sample from the patient; (b) measuring the [AAG] in the sample of the patient; (c) determining whether the sample has low [AAG], (d) classifying the patient as having an increased likelihood of responding therapeutically to the method of treating cancer if the sample has low [AAG], and (e) administering ARRY-520 to the patient classified as having an increased likelihood of response is provided.

In another aspect, a method for determining a higher likelihood of sensitivity to ARRY-520 therapy in a cancer patient comprising: (a) assaying a biological sample from the patient for [AAG]; and (b) identifying the patient as having a higher likelihood of sensitivity to ARRY-520 therapy when the biological sample is low in [AAG] is provided.

In another aspect, a method for determining a higher likelihood of sensitivity to ARRY-520 therapy in a cancer patient comprising: (a) obtaining a biological sample from the patient; (b) measuring the [AAG] in the biological sample; and (c) identifying the patient as having a higher likelihood of sensitivity to ARRY-520 therapy when the biological sample is low in [AAG] is provided.

In another aspect, a method of using ARRY-520 to treat a patient who has been diagnosed with levels of [AAG] of less than about 1.1 g/L, comprising administering one or more unit doses of ARRY-520.

In another aspect, a method of using ARRY-520 to treat a patient who has been diagnosed with levels of [AAG] of less than about 1.1 g/L, comprising administering one or more unit doses of ARRY-520 to said patient in amounts effective to produce a level of unbound ARRY-520 not less than the predicted in vitro IC₅₀.

In another aspect, a method of treating cancer in a patient having low [AAG], comprising administering to the patient an effective amount of ARRY-520 is provided.

In another aspect, a method of treating cancer in a mammal having low [AAG] comprising administering a therapeutically effective amount of ARRY-520 to the mammal is provided.

In another aspect, a method of treating a disease or disorder modulated by KSP, comprising administering to a mammal in need of such treatment an effective amount of ARRY-520, wherein the mammal has low [AAG] is provided.

In another aspect, use of ARRY-520 in the manufacture of a medicament for the treatment of cancer in a patient having low [AAG] is provided.

In another aspect, a pharmaceutical composition for treating a patient with cancer having low [AAG], comprising ARRY-520 is provided.

In another aspect, a pharmaceutical composition for treating a patient with cancer having low [AAG], comprising ARRY-520 and a pharmaceutically acceptable carrier or excipient is provided.

### BRIEF DESCRIPTION OF THE FIGURES

FIG. 1 shows a cellular assay.

FIG. 2 shows a Population PK (“popPK”) model simulation.

FIG. 3 shows a Population PK (“popPK”) model simulation.

FIG. 4 shows the analysis of human clinical trials.

FIG. 5 shows the analysis of human clinical trials.

FIG. 6 shows the variability of an assay.

FIG. 7 shows a linear regression comparing two assays.

FIG. 8 shows a linear regression comparing two assays.

FIG. 9 shows a linear regression comparing two assays.

FIG. 10 shows a linear regression comparing two assays.

### DETAILED DESCRIPTION OF THE INVENTION

Reference will now be made in detail to certain embodiments. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

Methods of this invention encompass methods of treating, preventing and/or managing various types of cancer and diseases and disorders associated with, or characterized by, undesired angiogenesis. As used herein, unless otherwise specified, the term “treating” or “treat” refers to the administration of a compound of the invention or other additional active agent after the onset of symptoms of the particular disease or disorder. The terms “treat” or “treatment” also refer to therapeutic or palliative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder. As used herein, unless otherwise specified, the term “preventing” refers to the administration prior to the onset of symptoms, particularly to patients at risk of cancer, and other diseases and disorders associated with, or characterized by, undesired angiogenesis. The term “prevention” includes the inhibition of a symptom of the particular disease or disorder. Patients with familial history of cancer and diseases and disorders associated with, or characterized by, undesired angiogenesis are preferred candidates for preventive regimens. As used herein and unless otherwise indicated, the term “managing” encompasses preventing the recurrence of the particular disease or disorder in a patient who had suffered from it, and/or lengthening the time a patient who had suffered from the disease or disorder remains in remission.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, skin cancer, including melanoma, as well as head and neck cancer.

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound described herein.

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The term “mammal” means a warm-blooded animal that has or is at risk of developing a disease described herein and includes, but is not limited to, guinea pigs, dogs, cats, rats, mice, hamsters, and primates, including humans.

Patients with Low [AAG]

ARRY-520 exhibits low micromolar affinity for AAG in in vitro assays, but not for other common serum proteins such as albumin (Example 1). It has been found that treating patients having a low [AAG] with ARRY-520 is beneficial.

The term “[AAG]” means the concentration of AAG as measured in a biological sample of a patient prior to administration of ARRY-520. The term “low [AAG]” means [AAG] less than about 1.1 g/L. As shown in Example 7, the 1.1 g/L level was measured in blood plasma using the R&D Systems Quantikine® assay. As shown in Example 8, there is variability in the assay of at least 8.6%. In certain embodiment, the term “about 1.1 g/L” means 1.1 g/L±20%. In certain embodiment, the term “about 1.1 g/L” means 1.1 g/L 10%. In certain embodiment, the term “about 1.1 g/L” means 1.1 g/L±8.6%. In another embodiment, low [AAG] means [AAG] less than about 1.1 g/L in blood plasma as determined in the R&D Systems Quantikine® assay (as described in Example 7).

It is also understood that various assays may be used to measure the [AAG]. Other assays may give slightly different results based on differences in that assay. If other assays are used, they should be correlated with the 1.1 g/L measurement of the R&D Systems Quantikine® assay used in Example 7. Scientific and statistical methods are known in the art for the correlation of two assays. Examples of correlations (cross comparisons) are shown in Example 10. Other assays may include, inter alia, the Randox Imola immunoturbidimetric, Randox Daytona immunoturbidimetric, Siemens Advia immunoturbidimetric and Siemens BNII immunonephelometric assays.

In certain embodiments, the biological sample that is used for measuring the [AAG] is blood. Drawing blood (obtaining a biological sample) from a patient is a well-known skill in the art. In a further embodiment, the biological sample that is used for measuring the [AAG] is plasma. In another further embodiment, the biological sample that is used for measuring the [AAG] is serum. In internal testing, there appeared to be a good correlation (>0.9) between serum and plasma [AAG] in the R&D Systems Quantikine®, Siemens Advia, Siemens BNII and Randox Imola assays (all assays were run per the manufacturer's protocols unless specified differently in the Examples).

ARRY-520 is typically administered intravenously. ARRY-520 is generally provided as a lyophilized powder contained in a Type 1 clear glass vial for IV use. The powder is reconstituted with sterile water for injection to form a solution and diluted with normal saline prior to IV administration.

The major dose limiting toxicity (“DLT”) of ARRY-520 has been found to be neutropenia. As such, prophylactic granulocyte colony-stimulating factory (“G-CSF”) may be administered.

ARRY-520 is generally administered on Days 1 and 2 of a 14 day cycle (Days 1 and 2 Q2W). ARRY-520 is generally administered on this schedule at 2.5 mg/m²/cycle (1.25 mg/m²/day) without G-CSF and 3.0 mg/m²/cycle (1.5 mg/m²/day) with prophylactic G-CSF. However, ARRY-520 may also be administered on Day 1 of a 14 day cycle (Day 1 Q2W) or Day 1 and 15 on a 28 day cycle (Days 1 and 15 Q4W).

It has been found that administering ARRY-520 to a patient having low [AAG] increases the likelihood of response of that patient to ARRY-520.

Accordingly, one embodiment provides ARRY-520 for use in treating cancer in a patient with low [AAG].

Certain embodiments provide ARRY-520 for use in treating cancer in a patient, comprising (a) assaying a biological sample from the patient for the [AAG], (b) determining whether the sample has low [AAG], and (c) administering a therapeutically effective amount of ARRY-520 to the patient if they have low [AAG].

Another embodiment provides ARRY-520 for use in treating cancer in a patient, comprising (a) obtaining a biological sample from the patient; (b) assaying the biological sample for the [AAG], (c) determining whether the sample has low [AAG], and (d) administering a therapeutically effective amount of ARRY-520 to the patient if they have low [AAG].

Certain embodiments provide a method for treating cancer in a cancer patient identified as having low [AAG] comprising a step of treating the patient with ARRY-520, comprising: (a) identifying the patient as having low [AAG] by assaying a biological sample from the patient, and (b) administering ARRY-520 to the patient having low [AAG].

Another embodiment provides a method for treating cancer in a cancer patient identified as having low [AAG] comprising a step of treating the patient with ARRY-520, comprising: (a) obtaining a biological sample from the patient; (b) identifying the patient as having low [AAG] by assaying the biological sample from the patient, and (c) administering ARRY-520 to the patient having low [AAG].

Certain embodiments provide a method of detecting a patient more likely to respond to ARRY-520, comprising obtaining a biological sample from the patient and assaying the sample to determine the [AAG], wherein low [AAG] is indicative of a patient more likely to respond to ARRY-520.

Another embodiment provides a method of detecting a patient more likely to respond to ARRY-520, comprising obtaining a biological sample from the patient, assaying the sample to determine the [AAG], and determining whether the patient is more likely to respond to ARRY-520, wherein low [AAG] is indicative of a patient more likely to respond to ARRY-520.

Certain embodiments provide a method for increasing the likelihood of response in a patient having cancer, comprising: (a) identifying the patient as having low [AAG] by assaying the biological sample from the patient; and (b) administering ARRY-520 to the patient classified as having an increased likelihood of response.

Another embodiment provides a method for increasing the likelihood of response in a patient having cancer, comprising: (a) obtaining a biological sample from the patient; (b) contacting the sample with an assay to measure the [AAG]; (c) determining whether the sample has low [AAG]; (d) classifying the patient as having an increased likelihood of response if the patient has low [AAG]; and (e) administering ARRY-520 to the patient classified as having an increased likelihood of response.

Certain embodiments provide a method for predicting an increased likelihood a patient will respond therapeutically to a method of treating cancer comprising administering ARRY-520, the method comprises: (a) measuring the [AAG] in a biological sample of the patient; (b) determining whether the sample has low [AAG], (c) classifying the patient as having an increased likelihood of responding therapeutically to the method of treating cancer if the sample has low [AAG], and (d) administering ARRY-520 to the patient classified as having an increased likelihood of response.

Another embodiment provides a method for predicting an increased likelihood a patient will respond therapeutically to a method of treating cancer comprising administering ARRY-520, the method comprises: (a) obtaining a biological sample from the patient; (b) measuring the [AAG] in the sample of the patient; (c) determining whether the sample has low [AAG], (d) classifying the patient as having an increased likelihood of responding therapeutically to the method of treating cancer if the sample has low [AAG], and (e) administering ARRY-520 to the patient classified as having an increased likelihood of response.

Certain embodiments provide a method for determining a higher likelihood of sensitivity to ARRY-520 therapy in a cancer patient comprising: (a) assaying a biological sample from the patient for [AAG]; and (b) identifying the patient as having a higher likelihood of sensitivity to ARRY-520 therapy when the biological sample is low in [AAG].

Certain embodiments provide a method for determining a higher likelihood of sensitivity to ARRY-520 therapy in a cancer patient comprising: (a) obtaining a biological sample from the patient; (b) measuring the [AAG] in the biological sample; and (c) identifying the patient as having a higher likelihood of sensitivity to ARRY-520 therapy when the biological sample is low in [AAG].

Certain embodiments provide a method of using ARRY-520 to treat a patient who has been diagnosed with levels of [AAG] of less than about 1.1 g/L, comprising administering one or more unit doses of ARRY-520.

Certain embodiments provide a method of using ARRY-520 to treat a patient who has been diagnosed with levels of [AAG] of less than about 1.1 g/L, comprising administering one or more unit doses of ARRY-520 to said patient in amounts effective to produce a level of unbound ARRY-520 not less than the predicted in vitro IC₅₀. In a further embodiment, the predicted in vitro IC₅₀ is about 0.2 ng/mL. In a further embodiment, the predicted in vitro IC₅₀ is 0.2 ng/mL.

Certain embodiments provide a method of treating cancer in a patient having low [AAG], comprising administering to the patient an effective amount of ARRY-520.

Certain embodiments provide a method of treating cancer in a mammal having low [AAG] comprising administering a therapeutically effective amount of ARRY-520 to the mammal.

Certain embodiments provide a method of treating a disease or disorder modulated by KSP, comprising administering to a mammal in need of such treatment an effective amount of ARRY-520, wherein the mammal has low [AAG].

Another embodiment provides the use of ARRY-520 in the manufacture of a medicament for the treatment of cancer in a patient having low AAG.

One embodiment includes a pharmaceutical composition for treating a patient with cancer having low [AAG] comprising ARRY-520. A further embodiment provides a pharmaceutical composition for treating a patient with cancer having low [AAG] comprising ARRY-520 together with a pharmaceutically acceptable carrier or excipient. In certain embodiments, the pharmaceutically acceptable excipient is mannitol.

Also provided are methods of treating a disease or condition by administering ARRY-520. In one embodiment, a human patient with low [AAG] is treated with ARRY-520, and a pharmaceutically acceptable carrier, adjuvant, or vehicle in an amount to detectably inhibit KSP activity.

In another embodiment, a method of treating or preventing cancer in a mammal in need of such treatment, wherein the method comprises administering to said mammal a therapeutically effective amount of ARRY-520.

In certain embodiments, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In certain embodiments, the cancer is a hematological cancer. In certain embodiments, the cancer is selected from lymphomas, leukemia and multiple myeloma. In certain embodiments, the cancer is selected from leukemia and multiple myeloma. In certain embodiments, the cancer is selected from acute myeloid leukemia and multiple myeloma. In certain embodiments, the cancer is multiple myeloma. In certain embodiments, the cancer is acute myeloid leukemia.

In certain embodiments, the cancer is a solid tumor. In certain embodiments, the cancer is selected from skin, breast, brain, cervical carcinoma, and testicular cancer. In certain embodiments, the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.

Combination Therapy

The compounds described herein and stereoisomers and pharmaceutically acceptable salts thereof may be employed alone or in combination with other therapeutic agents for treatment. The compounds described herein may be used in combination with one or more additional drugs, for example an anti-hyperproliferative (or anti-cancer) agent that works through action on a different target protein. The second compound of the pharmaceutical combination formulation or dosing regimen preferably has complementary activities to the compound described herein, such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

In certain embodiments, G-CSF is administered in combination with ARRY-520.

In certain embodiments, dexamethasone is administered in combination with ARRY-520. In certain embodiments, G-CSF is administered in combination with ARRY-520 and dexamethasone.

In certain embodiments, bortezomib is administered in combination with ARRY-520. In certain embodiments, G-CSF is administered in combination with ARRY-520 and bortezomib.

In certain embodiments, carfilzomib is administered in combination with ARRY-520. In certain embodiments, G-CSF is administered in combination with ARRY-520 and carfilzomib.

In certain embodiments, pomalidomide is administered in combination with ARRY-520. In certain embodiments, G-CSF is administered in combination with ARRY-520 and pomalidomide.

### EXAMPLES

For illustrative purposes, the following Examples are included. However, it is to be understood that these Examples do not limit the invention and are only meant to suggest a method of practicing the invention.

In the Examples described below, unless otherwise indicated all temperatures are set forth in degrees Celsius. Reagents were purchased from commercial suppliers, and were used without further purification unless otherwise indicated.

### Example 1

### ARRY-520 TRANSIL® Binding Methodology

TRANSIL® Binding Methodology (www.admecell.com)—Assay Buffers: phosphate buffered saline (“PBS”), pH 7.4 (Gibco 10010) and dimethylsulfoxide (“DMSO”). Plates: Ordered from ADMEcell (Alameda, Calif.). AGP—Full Plate: TBP-0211-0096; AGP—Strip Plate: TBP-0211-1196; HSA—Full Plate: TBP-0210-0096; HSA—Strip Plate: TBP-0210-1196. Stop Solution: 100% Acetonitrile spiked with Internal Standard (0.4 μM final concentration).

Drug Dilution: Dilution of drug samples to make a final 2 μM drug concentration (1% DMSO) (used 360 μL of final diluted drug per protein testing, i.e., 720 μL to run both AAG and HSA). Brought drug up to a 10 mM stock solution in DMSO. Diluted 10 mM stock to a 200 μM (0.2 mM) stock solution (added 4 μL of 10 mM stock solution into 196 μL, of DMSO). Diluted 200 μM stock to a 20 μM stock solution (added 100 μL of 200 μM DMSO stock solution into 900 μL of PBS Buffer, pH 7.4).

Thawed the assay kit either at room temperature (approximately 3 hours) or in the refrigerator at 4° C. overnight. The plates were put in the incubator, CO₂ (5%) for 30 minutes prior to addition of compound. A pre-dilution of the compounds (10-fold assay concentration) were prepared, see Drug Dilution step above. The drug dilutions were warmed in a 37° C. water bath prior to dosing compound into plate. Caution: Check carefully the pre-dilution for any precipitation and ensure a sufficient buffer solubility and stability of your test article. Test article solution (45 μL) was added to the tubes/well of the ready-to-use kit and incubated for 2 minutes. Mixed ten times by re-suspending the beads. Re-suspended approximately half of the total volume of the vial (total volume is 450 μL). Did not use orbital shakers. The vials were spun with a swing-out centrifuge for 10 minutes at 750 g. 100 μL was carefully transferred to 96-well plate. Acetonitrile (50 μL) was added with labetalol as the internal standard (0.4 μM final concentration). The plates were sealed for analysis. The concentration of the supernatant was quantified with LC/MS (API4000). See also, Brown, Karin D. supra. The result is in Table 1. pKa value was estimated using ACD/pKa DB software. Calculations:

$${K}_{d} = {c}_{free} \cdot \frac{1 - \frac{{c}_{total} - {c}_{free} \cdot {f}_{corr}}{{c}_{protein}}}{\frac{{c}_{total} - {c}_{free} \cdot {f}_{corr}}{{c}_{protein}}}$$

TABLE 1

| | Ionization | % Fu to | | | Fold |
|---|---|---|---|---|---|
| | State | Human | Kd (μM) - AVG | | Preference |
| Compound | (pKa) | Plasma | AAG | HSA | AAG vs. HSA |
| ARRY-520 | base (9.7) | 7 | 5 | 147 | 29 |

### Example 2

### 3 Day Cell (NCI H929 MM) Viability Assay

Cell Titer Blue Proliferation Assay: AAG effects on ARRY-520 cytotoxic activity in MM lines.

Reagents: RPMI-8226, H929, RPMI 1640 media, 10% FBS, Glutamax

Kit: Cell Titer-Blue Cell Viability Assay, Promega Corp. (Madison Wis.) Catalog No. G8081

Compound:
10 mM stock of ARRY-520 in DMSO
250 mg AAG from human plasma purchased from Sigma-Aldrich Co. LLC (St. Louis, Mo.) Catalog No. G9885
   Added 6.2 mL of PBS→40 mg/mL stock
   Stored at 4° C.

Procedure:
Setup ARRY-520 dilutions in a 96-well v-bottom plate:
   Placed 10 μL of DMSO in wells B2-B11
   Added 10 μL of 100 μM ARRY-520 to well B2 and mixed by pipetting up and down
   With a new pipette tip, transferred 10 μL of B2 to well B3 and mixed by pipetting up and down
   Repeated procedure through to B10, then discarded 10 μL from B10
   Well B11 is a DMSO control well
Wells B2-B10 now have 10 μL of 1:2 fold serial dilutions of ARRY-520 (50 μM to 200 nM)
Added 190 μL of growth medium to each well (1:20 dilution, 5% DMSO f.c.)
Placed lid on plate and placed to the side.
Prepared AAG solutions:
   30 mg/mL: 150 μL of 40 mg/mL+50 μL PBS
   20 mg/mL: 100 μL of 40 mg/mL+100 μL PBS
   10 mg/mL: 100 μL of 40 mg/mL+300 μL PBS
   5 mg/mL: 100 μL of 10 mg/mL+100 μL PBS
Counted MM cells
Placed 1.2×10⁶ cells in 8 mL of growth media and mixed gently to create a cell suspension (143 cells/4)
Added 10 μL ( 1/10 volume) of compound dilutions (above) to two 96-well, black walled, clear bottom, tissue culture plates (Sigma-Aldrich Catalog No. CLS3904) as outlined below. Made sure that the 10 μL is on the bottom of each plate
Placed 10 μL of ARRY-520 dilutions from wells B2-B11 of v-bottom plate into rows B-G of 2 identical black-walled 96 well plates
Added 80 μL of 150 cells/4 cell suspension (12,000 cells/well total) to columns 2-11 of rows B-G: Plate 1—RPMI 8226 and Plate 2—NCI H929
AAG addition:
   Added 10 μL of PBS to row B of each plate
   Added 10 μL of 40 mg/mL AAG solution to Row C of each plate (4 mg/mL f.c.)
   Added 10 μL of 30 mg/mL AAG solution to Row D of each plate (3 mg/mL f.c.)
   Added 10 μL of 20 mg/mL AAG solution to Row E of each plate (2 mg/mL Lc.)
   Added 10 μL of 10 mg/mL AAG solution to Row F of each plate (1 mg/mL f.c.)
   Added 10 μL of 5 mg/mL AAG solution to Row G of each plate (0.5 mg/mL f.c.)
Added 100 μL of media to remaining outer wells of plates
After cells seeded with compound in a final volume of 100 μL, plates were placed in 37° C. incubators (5% or 0% CO₂) and assayed 24, 48 and 72 hours later:
   20 μL of Cell Titer Blue reagent was added to each of the wells, mixed briefly on a plate shaker for 10 seconds, and placed back in the incubator for 2-4 hours
Fluorescence was read on Gemini (fluorescent plate reader, Spectramax, Molecular Devices) at Ex/Em 560/590 nm, with 590 nm cut-off filter
The calculated fluorescent signal minus background (RFU) was exported to Excel and used to analyze the dose response with XLFit4 with a 4-parameter fit equation: Fit=(A+((B−A)/(1+((C/x)̂D))))

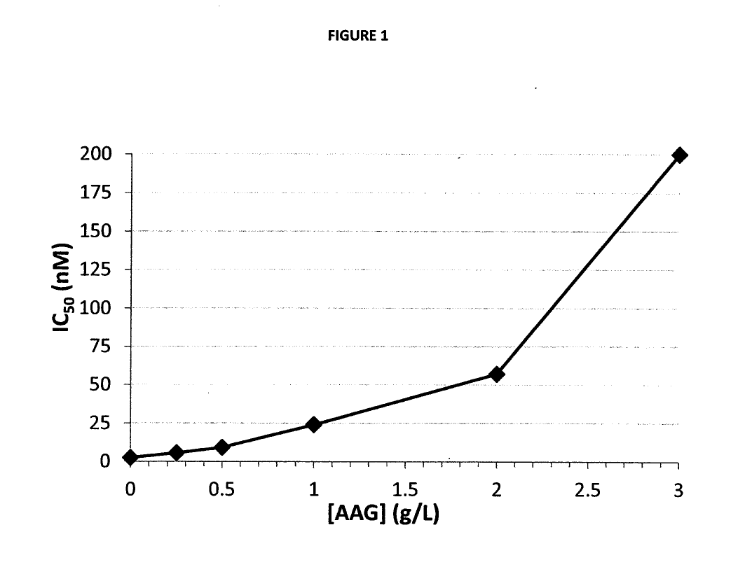

Results: Increasing AAG levels clearly decrease cell sensitivity to ARRY-520 in both cell lines. Cellular Sensitivity to ARRY-520 decreases greater than 30 fold as [AAG] increases in the assay, which is consistent with reduced unbound ARRY-520 with increasing [AAG]. See FIG. 1 for NCI H929 results.

### Example 3

### A Study of ARRY-520 in Patients with Advanced Cancer

Phase 1 Study of ARRY-520. See clinicaltrials.gov/ct2/show/NCT00462358; and Goncalves, P., et al., “A Phase 1 Safety and Pharmacokinetic Study of ARRY-520 in Solid Tumors”, 2010 American Society of Clinical Oncology Annual Meeting, Abstract #2570, www.arraybiopharma.com/_documents/Publication/PubAttachment387.pdf, the contents of which are herein incorporated by reference in their entirety.

### Example 4

### A Study of ARRY-520 in Patients with Advanced Myeloid Leukemia

Phase 2 Study of ARRY-520 as a Single Agent. See clinicaltrials.gov/ct2/show/NCT00637052; Garcia-Manero, Guillermo, et al., “A Phase 1 Dose-Escalation Study of the Novel KSP Inhibitor ARRY-520 in Advanced Leukemias”, 2009 51st American Society of Hematology Annual Meeting and Exposition, Abstract #22799, www.arraybiopharma.com/_documents/Publication/PubAttachment368.pdf; and Estrov, Z., et al., “A Phase 1 Dose-Escalation Study of the Novel KSP Inhibitor ARRY-520 in Advanced Leukemias”, 2009 American Society of Clinical Oncology Annual Meeting, www.arraybiopharma.com/_documents/Publication/PubAttachment347.pdf, the contents of which are herein incorporated by reference in their entirety.

### Example 5

### A Study of ARRY-520 in Patients with Relapsed or Refractory Multiple Myeloma

Phase ½ Study of ARRY-520 and Dexamethasone. See clinicaltrials.gov/ct2/show/NCT00821249; Shah, J. J., et al., “A Phase ½ Trial of the KSP Inhibitor ARRY-520 in Relapsed/Refractory Multiple Myeloma”, 2010 American Society of Hematology Meeting, Publication Number 1959, www.arraybiopharma.com/_documents/Publication/PubAttachment428.pdf; Shah, J. J., et al., “ARRY-520 Shows Durable Responses in Patients with Relapsed/Refractory Multiple Myeloma in a Phase 1 Dose-Escalation Study”, 2011 American Society of Hematology Annual Meeting, www.arraybiopharma.com/_documents/Publication/PubAttachment493.pdf; Lonial, S., et al., “The Novel KSP Inhibitor ARRY-520 Demonstrates Single-Agent Activity in Refractory Myeloma: Results From a Phase 2 Trial in Patients with Relapsed/Refractory Multiple Myeloma”, 2011 American Society of Hematology Annual Meeting, Abstract #2935, www.arraybiopharma.com/_documents/Publication/PubAttachment563.pdf; Shah, J. J., et al., “The Novel KSP Inhibitor ARRY-520 Is Active Both with and without Low-Dose Dexamethasone in Patients with Multiple Myeloma Refractory to Bortezomib and Lenalidomide: Results From a Phase 2 Study”, 2012 American Society of Hematology Meeting, www.arraybiopharma.com/_documents/Publication/PubAttachment556.pdf; Lonial, S., et al., “The Novel KSP Inhibitor ARRY-520 Demonstrates Single-Agent Activity in Refractory Myeloma: Results From a Phase 2 Trial in Patients with Relapsed/Refractory Multiple Myeloma”, 2011 American Society of Hematology Annual Meeting, Abstract 2935, www.arraybiopharma.com/_documents/Publication/PubAttachment563.pdf; and Lonial, Sagar, et al., “Single-Agent Activity of the Novel KSP Inhibitor ARRY-520 in Patients with Relapsed or Refractory Multiple Myeloma (RRMM): Results from Subgroup Analyses.” 2013 International Myeloma Workshop, Poster # P-224, www.arraybiopharma.com/_documents/Publication/PubAttachment563.pdf, the contents of which are herein incorporated by reference in their entirety.

### Example 6

### PK Modeling

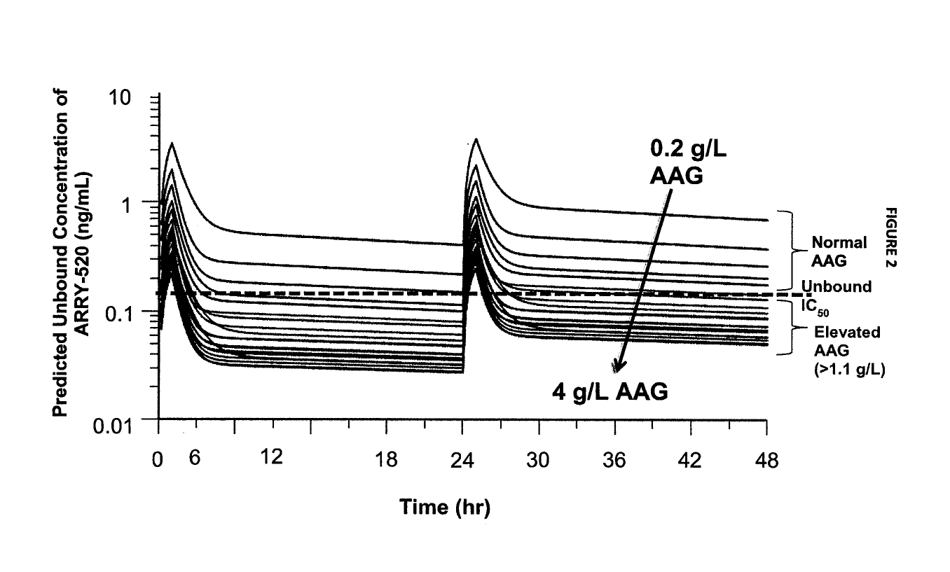

Population PK (“popPK”) modeling was based on plasma concentrations of ARRY-520 from patients in Examples 3 to 5. Model optimization was accomplished using the QRPEM engine of Phoenix 6.3 (Pharsight Corporation, St. Louis, Mo.). Model selection was based on AIC comparisons and diagnostic plots. Simulations were for a typical patient receiving Phase 2 dose of 1.5 mg/m² on day 1 and day 2 with varying [AAG]. See FIG. 2. At [AAG] greater than 1.1 g/L, sustained exposure above the estimated in vitro unbound IC₅₀ is not predicted.

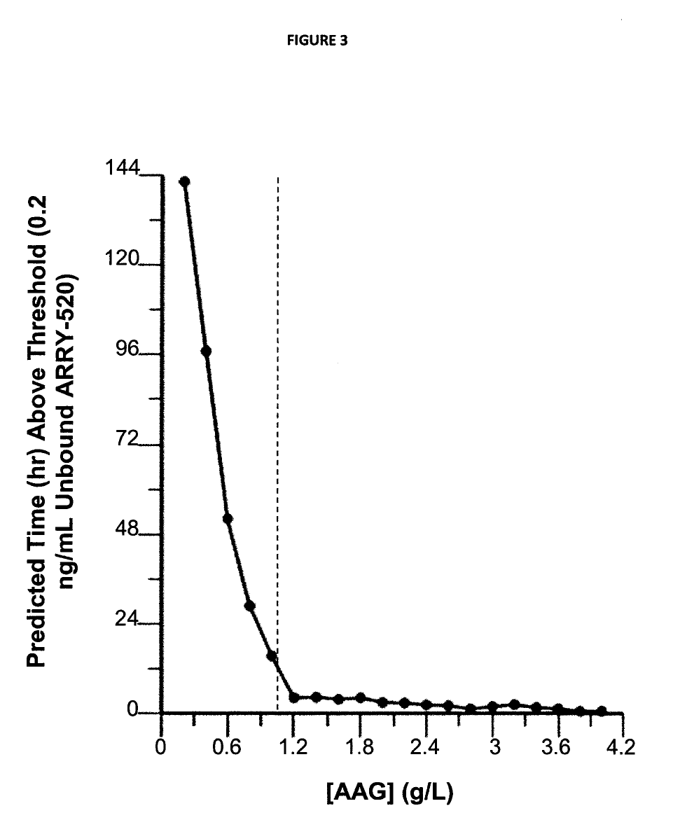

The predicted inhibition of KSP is not sustained when AAG is elevated. Prolonged inhibition of KSP (greater than 24 hours above the unbound ARRY-520 IC₅₀ of 0.2 ng/mL) may be needed for clinical activity. Unbound concentration of ARRY-520/time was simulated vs. [AAG] based on the popPK model (N=50 patients per AAG level). The predicted total time of concentration unbound ARRY-520 greater than 0.2 ng/mL was calculated. See FIG. 3.

### Example 7

### AAG Assay

The R&D Systems, Inc. (Minneapolis, Minn.) Quantikine® Human α1-Acid Glycoprotein Immunoassay (Catalog No. DAGPOO) is a 4.5 hour solid-phase ELISA designed to measure human AAG in cell culture supemates, serum, plasma, and urine. This assay employed the quantitative sandwich enzyme immunoassay technique. A monoclonal antibody specific for AAG has been pre-coated onto a microplate. Standards and samples are pipetted into the wells, and any AAG present is bound by the immobilized antibody. After washing away any unbound substances, an enzyme-linked polyclonal antibody specific for AAG is added to the wells. Following a wash to remove any unbound antibody-enzyme reagent, a substrate solution is added to the wells, and color develops in proportion to the amount of AAG bound in the initial step. The color development is stopped, and the intensity of the color is measured. The assay was performed as described in the package insert, except where described otherwise below.

Materials provided in the R&D Systems, Inc. Quantikine® Human AAG Immunoassay (Catalog No. DAGPOO) include:
AAG Microplate (Part 893786)—96 well polystyrene microplate (12 strips of 8 wells) coated with a mouse monoclonal antibody against AAG.
AAG Conjugate (Part 893787)—21 mL of polyclonal antibody against AAG conjugated to horseradish peroxidase with preservatives.
AAG Standard (Part 893788)—3 vials (400 ng/vial) of human AAG in a buffer with preservatives; lyophilized.
Assay Diluent RD1-73 (Part 895541)—12.5 mL of a buffer with preservatives.
Calibrator Diluent RD5-20 Concentrate (Part 895346)—2 vials (21 mL/vial) of a buffered protein base with preservatives.
Wash Buffer Concentrate (Part 895003)—21 mL of a 25-fold concentrated solution of buffered surfactant with preservatives.
Color Reagent A (Part 895000)—12.5 mL of stabilized hydrogen peroxide. Color Reagent B (Part 895001)—12.5 mL of stabilized chromogen (tetramethylbenzidine).
Stop Solution (Part 895032)—6 mL of 2 N sulfuric acid.
Plate Covers—4 adhesive strips

Other material required for R&D Systems, Inc. Quantikine® Human AAG Immunoassay (Catalog No. DAGP00) include:
Microplate reader capable of measuring absorbance at 450 nm, with the correction wavelength set at 540 nm or 570 nm
Pipettes and pipette tips.
Deionized or distilled water
Squirt bottle, manifold dispenser, or automated microplate washer
500 mL graduated cylinder
2-8° C. incubator
Test tubes for dilution of standards and samples

Sample collection and storage: Serum—Used a serum separator tube (SST) and allowed samples to clot for 30 minutes before centrifugation for 15 minutes at 1000×g. Removed serum and assay immediately or aliquot and store samples at ≦−20° C. Avoided repeated freeze-thaw cycles. Plasma—Collected plasma using EDTA or heparin as an anticoagulant. Centrifuged for 15 minutes at 1000×g within 30 minutes of collection. Assayed immediately or aliquoted and stored samples at ≦−20° C. Avoided repeated freeze-thaw cycles. Note: Citrate plasma is not validated for use in this assay.

Sample Preparation: Serum and plasma samples required a 10,000-fold dilution. A suggested 10,000-fold dilution may be accomplished by adding 10 μL of sample to 990 μL of Calibrator Diluent RD5-20 (1×). Completed the 10,000-fold dilution by adding 10 μL of diluted sample to 990 μL of Calibrator Diluent RD5-20 (1×).

Reagent Preparation (all reagents were brought to room temperature before use):

Wash Buffer—If crystals have formed in the concentrate, warmed to room temperature and mixed gently until the crystals were completely dissolved. Diluted 20 mL of Wash Buffer Concentrate into deionized or distilled water, to prepare 500 mL of Wash Buffer.

Calibrator Diluent RD5-20 (1×)—Added 20 mL of Calibrator Diluent RD5-20 Concentrate to 80 mL of deionized or distilled water, to prepare 100 mL of Calibrator Diluent RD5-20 (1×).

Substrate Solution—Color Reagents A and B should be mixed together in equal volumes within 15 minutes of use. Protect from light. 200 μL of the resultant mixture was required per well.

AAG Standard—Reconstituted the AAG Standard with 0.5 mL of Calibrator Diluent RD5-20 (1×). This reconstitution produced a stock solution of 800 ng/mL. Mixed the standard to ensure complete reconstitution, and allowed the standard to sit for a minimum of 15 minutes with gentle agitation prior to making dilutions.

Pipetted 250 μL of Calibrator Diluent RD5-20 (1×) into each tube. Used the AAG stock solution to produce a 2-fold dilution series. Mixed each tube thoroughly before the next transfer. The 800 ng/mL standard served as the high standard. Calibrator Diluent RD5-20 (1×) served as the zero standard (0 ng/mL).

Procedure: All reagents and samples were brought to room temperature before use. It was recommended that all samples and standards be assayed at least in duplicate.
1. Prepared all reagents, working standards, and samples as described above.
2. Removed excess microplate strips from the plate frame, returned them to the foil pouch containing the desiccant pack, and resealed.
3. Added 100 μL of Assay Diluent RD1-73 to each well.
4. Added 50 μL of Standard or sample per well (serum and plasma samples required dilution as described above). Covered with the adhesive strip provided. Incubated for 2 hours at room temperature. A plate layout was provided to record standards and samples assayed.
5. Aspirated each well and washed, repeating the process three times for a total of four washes. Washed by filling each well with Wash Buffer (400 μL) using a squirt bottle, manifold dispenser, or autowasher. Completed removal of liquid at each step was essential to good performance. After the last wash, removed any remaining Wash Buffer by aspirating or decanting. Inverted the plate and blot it against clean paper towels.
6. Added 200 μL of AAG Conjugate to each well. Covered with a new adhesive strip. Incubated for 2 hours at room temperature.
7. Repeated the aspiration/wash as in step 5.
8. Added 200 μL of Substrate Solution to each well. Incubated for 30 minutes at room temperature. Protected from light.
9. Added 50 μL of Stop Solution to each well. The color in the wells should change from blue to yellow. If the color in the wells was green or the color change did not appear uniform, gently tapped the plate to ensure thorough mixing.
10. Determined the optical density of each well within 30 minutes, using a microplate reader set to 450 nm. If wavelength correction was available, set to 540 nm or 570 nm. If wavelength correction was not available, subtracted readings at 540 nm or 570 nm from the readings at 450 nm. This subtraction corrected for optical imperfections in the plate. Readings made directly at 450 nm without correction may be higher and less accurate.

Calculation of Results: Averaged the duplicate readings for each standard, control, and sample, and subtracted the average zero standard optical density value. Created a standard curve by reducing the data using computer software capable of generating a four parameter logistic (4-PL) curve-fit. As an alternative, constructed a standard curve by plotting the mean absorbance for each standard on the y-axis against the concentration on the x-axis and drew a best fit curve through the points on the graph. The data may be linearized by plotting the log of the AAG concentrations versus the log of the O.D., and the best fit line can be determined by regression analysis. This procedure produced an adequate, but less precise fit of the data. If samples have been diluted, the concentration read from the standard curve must be multiplied by the dilution factor.

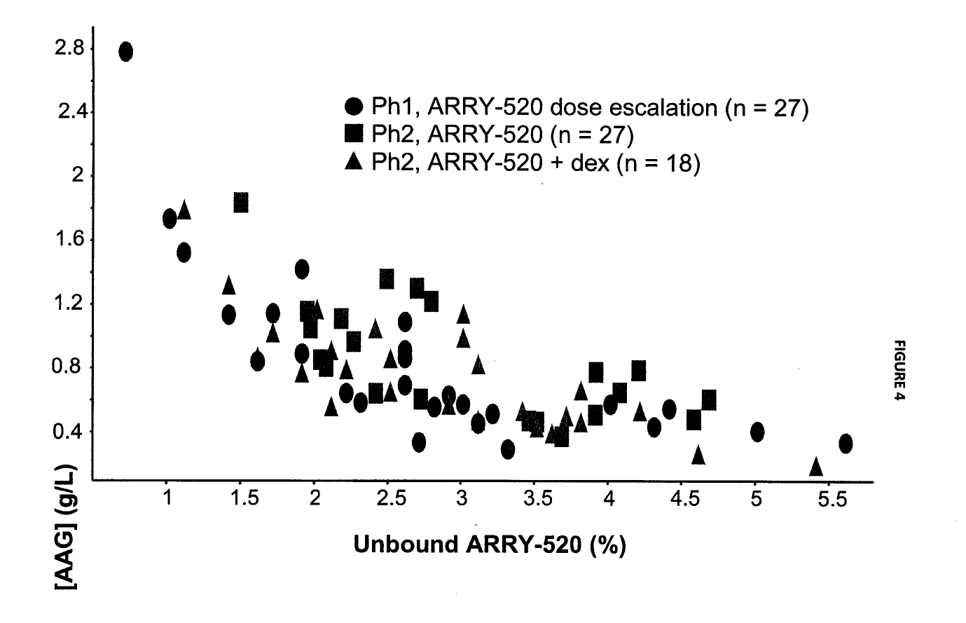

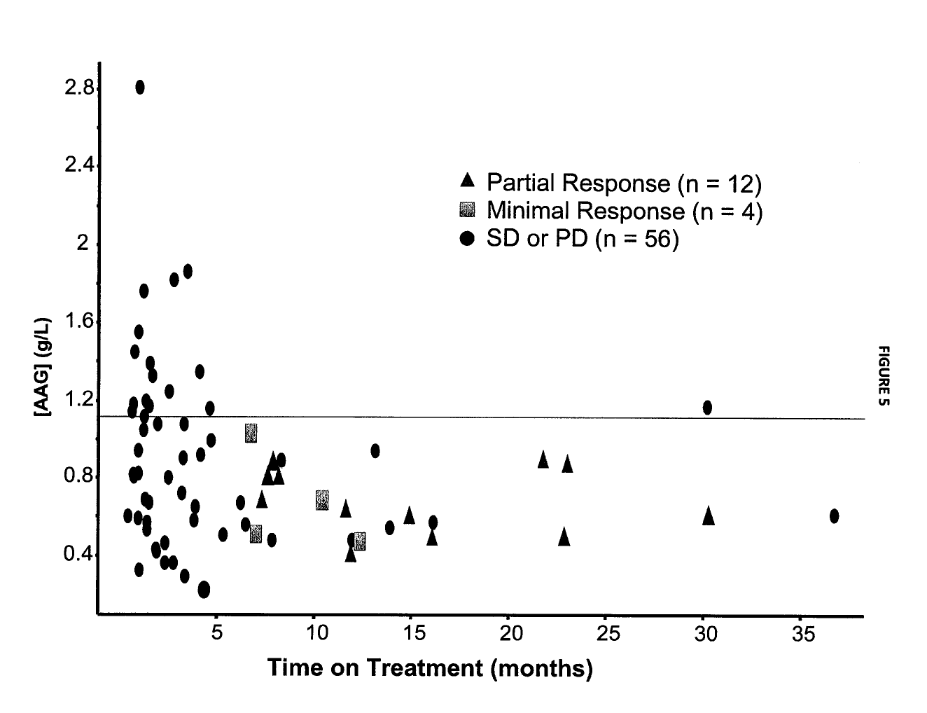

Ex vivo analysis of unbound ARRY-520 and [AAG] in baseline multiple myeloma patient plasma samples (from Example 5) is shown in FIG. 4. Patient plasma samples with higher baseline [AAG] correlated with lower available ARRY-520. Elevated [AAG] predicts a lack of response and decreased time on treatment as shown in FIG. 5. Most patients with “high” baseline [AAG] (greater than 1.1 g/L) came off study in less than 5 months. [AAG] for all patients achieving a clinical response (minimal response (“MR”) or partial response (“PR”)) was below 1.1 g/L at screening. The risk for discontinuing the study increases 2.5-fold with each 1.0 g/L increase in [AAG] (p<0.01). Excluding patients with baseline [AAG] above an arbitrary cut off of 1.1 g/L results in an increased response rate and time on study following ARRY-520 treatment in all studies as shown in Table 2. All patients responding were below [AAG] of 1.1 g/L at baseline (p=0.03; FET, 2-tailed). Approximately 30% of patients had [AAG] greater than 1.1 g/L at baseline.

TABLE 2

| | Phase 1 | | Phase 2 Single Agent | | Phase 2 + dex | |
|---|---|---|---|---|---|---|
| | All | [AAG] | All | [AAG] | All | [AAG] |
| | Pa- | <1.1 | Pa- | <1.1 | Pa- | <1.1 |
| | tients | g/L | tients | g/L | tients | g/L |
| Median | 7 | 6 | 7 | 6 | 10 | 10 |
| Prior | | | | | | |
| Regimens | | | | | | |
| n (evalu- | 27 | 20 | 27 | 21 | 18 | 12 |
| able) | | | | | | |
| ≧PR | 3 (11%) | 3 (15%) | 5 (19%) | 5 (24%) | 4 (22%) | 4 (33%) |
| ≧MR | 4 (15%) | 4 (20%) | 6 (22%) | 6 (29%) | 6 (33%) | 6 (50%) |
| Median | 3.9 | 5.2 | 2.8 | 3.4 | 3.9 | 6.2 |
| Time on | | | | | | |
| Study | | | | | | |
| (months) | | | | | | |

### Example 8

### Variability of AAG Assay

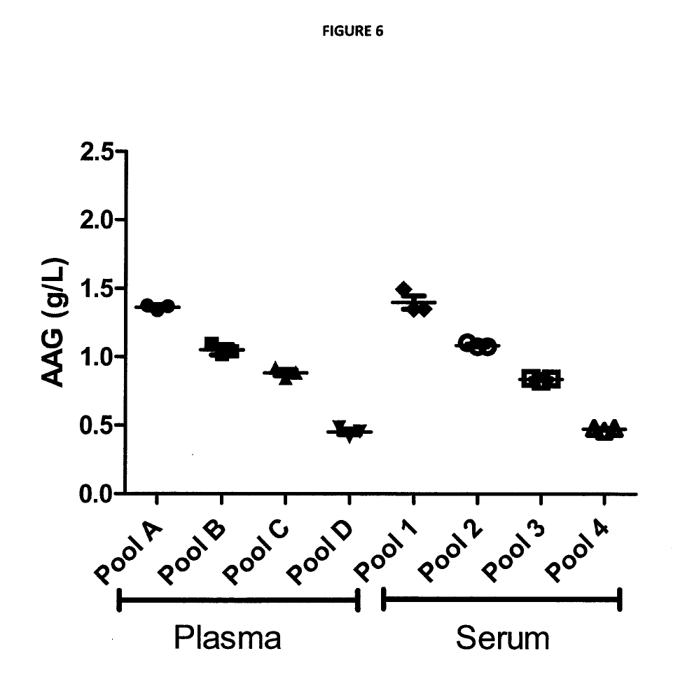

Four samples were run in the R&D Systems, Inc. Quantikine® Human α1-Acid Glycoprotein Immunoassay of Example 7 in duplicate over 3 non-consecutive days to assess variability around the preliminary cut point. CV's ranged from 1.7% to 8.6%. The results are shown in FIG. 6.

### Example 9

### AAG Assay

The Randox Laboratories Ltd. (Crumlin, United Kingdom) Immunoturbidimetric Protein Diagnostic Reagent for Alpha-1-Acid Glycoprotein RX Series 2472 was used on RX Imola series instrument. The assay was performed as described in the package insert, except where described otherwise below.

Reagent Composition:

| | Initial Concentration |
|---|---|
| R1. Assay Buffer | |
| Polyethylene Glycol | maximum 6% |
| Tris/HCl buffer | 20 mmol/l, pH 7.4 |
| Sodium Chloride | 150 mmol/l |
| Sodium Azide | |
| R2. Antibody Reagent | |
| Anti (human) alpha-1-acid glycoprotein | |
| Tris/HCl buffer | 20 mmol/l, pH 7.4 |
| Sodium Chloride | 150 mmol/l |
| Sodium Azide | |

Material Provided: R1 Assay Buffer and R2 Antibody Reagent.

Materials Required: 0.9% NaCl Solution, Randox Liquid Specific Protein Calibrator (Catalog No. IT 2692), Randox Liquid Assayed Specific Protein Controls Level 1 (Catalog No. PS 2682), Level 2 (Catalog No. PS 2683), and Level 3 (Catalog No. PS 2684).

Procedure: Entered lot specific values given in the specific protein calibrator insert. The Chemistry parameter for Randox Dedicated RX series Assays are predefined on the hard drive of the analyzer PC. The required programs should be downloaded to the analyzer software. All necessary instructions are encoded on the bar code. Randox Liquid Assayed Specific Protein Calibrator was used for calibration. Randox Liquid Assayed Specific Protein Controls, Level 1, Level 2 and Level 3 were used for daily quality control.

### Example 10

### Comparison of Assays

The R&D Systems, Inc. Quantikine® Human α1-Acid Glycoprotein Immunoassay of Example 7 was compared against the Siemens BNII assay (performed as per manufacturer's protocol) and the Randox Imola assay (performed as per manufacturer's protocol except as different in Example 9). The values were compared across assays and serum and plasma within each assay.

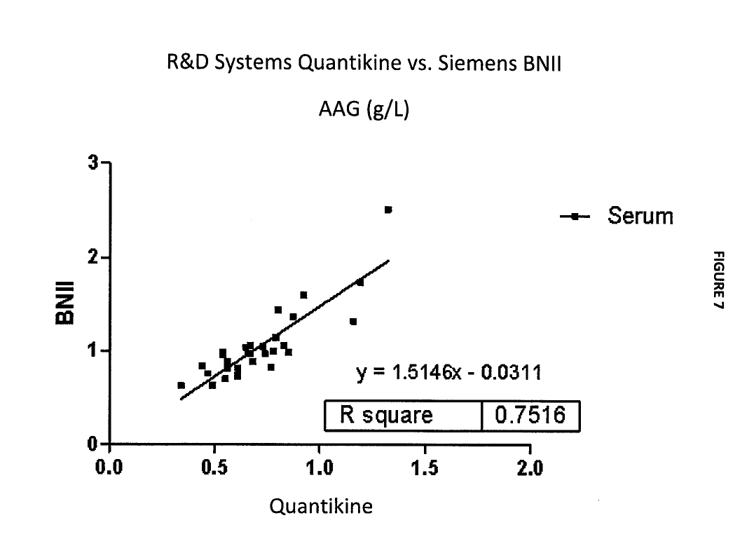

Comparison testing was done in serum samples in 20 MM and 10 healthy volunteers in the R&D Systems Quantikine® and Siemens BNII assays. Using this linear regression, the 1.1 g/L cut-off in the R&D Systems Quantikine® assay was calculated to be 1.635 g/L using the Siemens BNII assay. The results are shown in FIG. 7 and Table 3.

TABLE 3

| Sample # | R&D | BNII | % Difference |
|---|---|---|---|
| MM1 | 0.67 | 1.06 | 45.09% |
| MM2 | 0.66 | 0.98 | 39.02% |
| MM3 | 0.61 | 0.82 | 29.37% |
| MM4 | 1.32 | 2.52 | 62.50% |
| MM5 | 1.19 | 1.74 | 37.54% |
| MM6 | 0.54 | 0.96 | 56.00% |
| MM7 | 0.73 | 1.05 | 35.96% |
| MM8 | 0.87 | 1.37 | 44.64% |
| MM9 | 0.65 | 1.04 | 46.15% |
| MM10 | 0.78 | 1.00 | 24.72% |
| MM11 | 0.34 | 0.63 | 59.79% |
| MM12 | 0.67 | 0.97 | 36.59% |
| MM13 | 0.79 | 1.15 | 37.11% |
| MM14 | 0.44 | 0.84 | 62.50% |
| MM15 | 0.92 | 1.60 | 53.97% |
| MM16 | 0.74 | 0.97 | 26.90% |
| MM17 | 1.16 | 1.32 | 12.90% |
| MM18 | 0.56 | 0.89 | 45.52% |
| MM19 | 0.83 | 1.06 | 24.34% |
| MM20 | 0.55 | 0.70 | 24.00% |
| H1 | 0.77 | 0.83 | 7.50% |
| H2 | 0.56 | 0.81 | 36.50% |
| H3 | 0.8 | 1.44 | 57.14% |
| H4 | 0.68 | 0.89 | 26.75% |
| H5 | 0.61 | 0.74 | 19.26% |
| H6 | 0.61 | 0.73 | 17.91% |
| H7 | 0.54 | 0.99 | 58.82% |
| H8 | 0.47 | 0.76 | 47.15% |
| H9 | 0.85 | 0.99 | 15.22% |
| H10 | 0.49 | 0.63 | 25.00% |

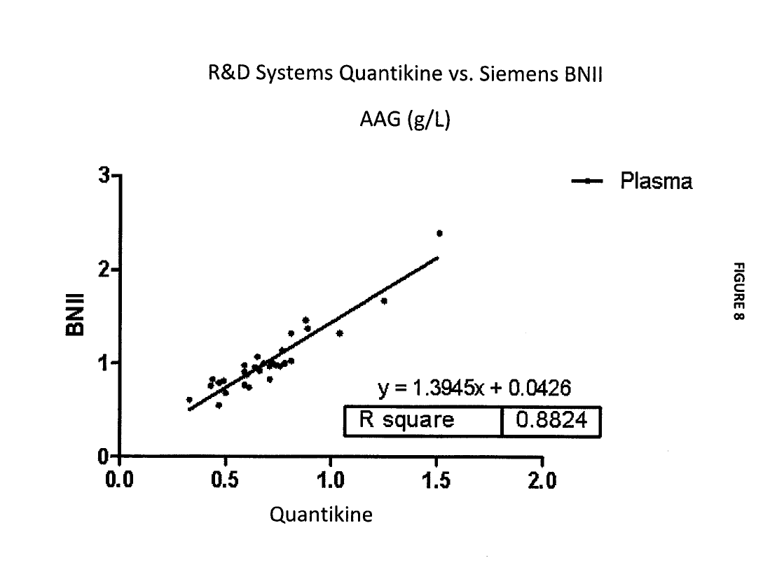

Comparison testing was done in plasma samples in 20 MM and 10 healthy volunteers in the R&D Systems Quantikine® and Siemens BNII assays. Using this linear regression, the 1.1 g/L cut-off in the R&D Systems Quantikine® assay was calculated to be 1.577 g/L using the Siemens BNII assay. The results are shown in FIG. 8 and Table 4.

TABLE 4

| Sample # | R&D | BNII | % Difference |
|---|---|---|---|
| MM1 | 0.68 | 1.00 | 38.10% |
| MM2 | 0.72 | 1.01 | 33.53% |
| MM3 | 0.49 | 0.81 | 49.23% |
| MM4 | 1.51 | 2.40 | 45.52% |
| MM5 | 1.25 | 1.67 | 28.77% |
| MM6 | 0.59 | 0.98 | 49.68% |
| MM7 | 0.76 | 0.97 | 24.28% |
| MM8 | 0.89 | 1.37 | 42.48% |
| MM9 | 0.65 | 1.07 | 48.84% |
| MM10 | 0.74 | 0.98 | 27.91% |
| MM11 | 0.33 | 0.61 | 59.57% |
| MM12 | 0.66 | 0.92 | 32.91% |
| MM13 | 0.77 | 1.14 | 38.74% |
| MM14 | 0.44 | 0.83 | 61.42% |
| MM15 | 0.88 | 1.46 | 49.57% |
| MM16 | 0.71 | 0.97 | 30.95% |
| MM17 | 1.04 | 1.32 | 23.73% |
| MM18 | 0.59 | 0.91 | 42.67% |
| MM19 | 0.81 | 1.03 | 23.91% |
| MM20 | 0.5 | 0.68 | 30.51% |
| H1 | 0.71 | 0.83 | 15.58% |
| H2 | 0.47 | 0.79 | 50.79% |
| H3 | 0.81 | 1.32 | 47.89% |
| H4 | 0.6 | 0.88 | 37.84% |
| H5 | 0.61 | 0.74 | 19.26% |
| H6 | 0.59 | 0.77 | 26.47% |
| H7 | 0.64 | 0.96 | 40.00% |
| H8 | 0.43 | 0.76 | 55.46% |
| H9 | 0.78 | 1.00 | 24.72% |
| H10 | 0.47 | 0.55 | 15.69% |

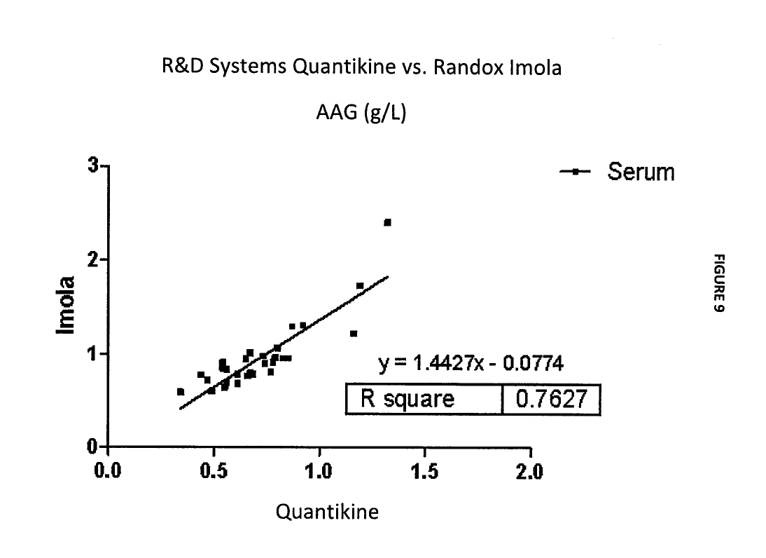

Comparison testing was done in serum samples in 20 MM and 10 healthy volunteers in the R&D Systems Quantikine® and Randox Imola assays. Using this linear regression, the 1.1 g/L cut-off in the R&D Systems Quantikine® assay was calculated to be 1.510 g/L using the Randox Imola assay. The results are shown in FIG. 9 and Table 5.

TABLE 5

| Sample # | R&D | Imola | % Difference |
|---|---|---|---|
| MM1 | 0.67 | 1.0144 | 40.89% |
| MM2 | 0.66 | 0.7632 | 14.50% |
| MM3 | 0.61 | 0.7842 | 24.99% |
| MM4 | 1.32 | 2.4052 | 58.26% |
| MM5 | 1.19 | 1.7283 | 36.89% |
| MM6 | 0.54 | 0.8491 | 44.50% |
| MM7 | 0.73 | 0.9776 | 29.00% |
| MM8 | 0.87 | 1.2995 | 39.59% |
| MM9 | 0.65 | 0.9467 | 37.16% |
| MM10 | 0.78 | 0.9102 | 15.41% |
| MM11 | 0.34 | 0.59 | 53.76% |
| MM12 | 0.67 | 0.7906 | 16.51% |
| MM13 | 0.79 | 0.9579 | 19.21% |
| MM14 | 0.44 | 0.7758 | 55.24% |
| MM15 | 0.92 | 1.309 | 34.90% |
| MM16 | 0.74 | 0.8935 | 18.79% |
| MM17 | 1.16 | 1.2222 | 5.22% |
| MM18 | 0.56 | 0.8364 | 39.59% |
| MM19 | 0.83 | 0.9556 | 14.07% |
| MM20 | 0.55 | 0.6469 | 16.19% |
| H1 | 0.77 | 0.8067 | 4.66% |
| H2 | 0.56 | 0.6945 | 21.44% |
| H3 | 0.8 | 1.063 | 28.23% |
| H4 | 0.68 | 0.7849 | 14.32% |
| H5 | 0.61 | 0.6837 | 11.39% |
| H6 | 0.61 | 0.6834 | 11.35% |
| H7 | 0.54 | 0.9057 | 50.59% |
| H8 | 0.47 | 0.7162 | 41.51% |
| H9 | 0.85 | 0.9556 | 11.70% |
| H10 | 0.49 | 0.601 | 20.35% |

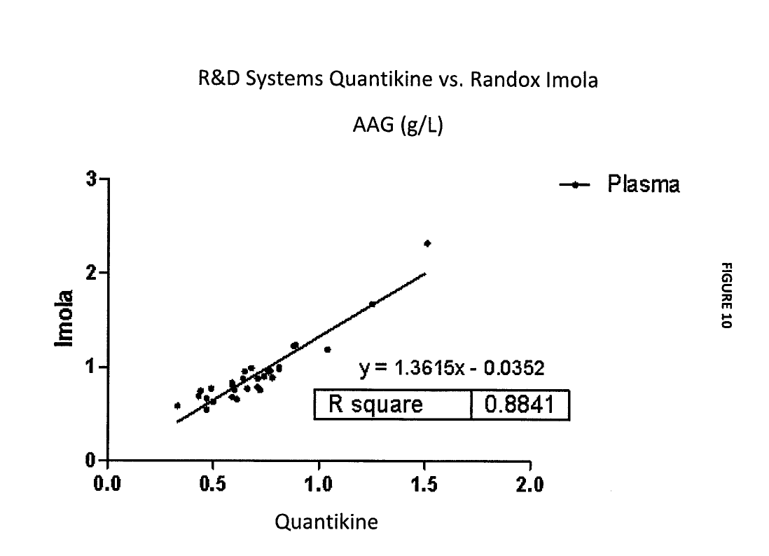

Comparison testing was done in plasma samples in 20 MM and 10 healthy volunteers in the R&D Systems Quantikine® and Randox Imola assays. Using this linear regression, the 1.1 g/L cut-off in the R&D Systems Quantikine® assay was calculated to be 1.462 g/L using the Randox Imola assay. The results are shown in FIG. 10 and Table 6.

TABLE 6

| Sample # | R&D | Imola | % Difference |
|---|---|---|---|
| MM1 | 0.68 | 0.9916 | 37.28% |
| MM2 | 0.72 | 0.7535 | 4.55% |
| MM3 | 0.49 | 0.7717 | 44.65% |
| MM4 | 1.51 | 2.3279 | 42.62% |
| MM5 | 1.25 | 1.6753 | 29.08% |
| MM6 | 0.59 | 0.8318 | 34.01% |
| MM7 | 0.76 | 0.9486 | 22.08% |
| MM8 | 0.89 | 1.2362 | 32.57% |
| MM9 | 0.65 | 0.9517 | 37.67% |
| MM10 | 0.74 | 0.8981 | 19.30% |
| MM11 | 0.33 | 0.5866 | 55.99% |
| MM12 | 0.66 | 0.7685 | 15.19% |
| MM13 | 0.77 | 0.9596 | 21.92% |
| MM14 | 0.44 | 0.7462 | 51.63% |
| MM15 | 0.88 | 1.2265 | 32.90% |
| MM16 | 0.71 | 0.877 | 21.05% |
| MM17 | 1.04 | 1.1887 | 13.34% |
| MM18 | 0.59 | 0.8074 | 31.11% |
| MM19 | 0.81 | 0.9816 | 19.16% |
| MM20 | 0.5 | 0.6286 | 22.79% |
| H1 | 0.71 | 0.7843 | 9.94% |
| H2 | 0.47 | 0.6642 | 34.24% |
| H3 | 0.81 | 0.999 | 20.90% |
| H4 | 0.6 | 0.7558 | 22.98% |
| H5 | 0.61 | 0.658 | 7.57% |
| H6 | 0.59 | 0.6798 | 14.14% |
| H7 | 0.64 | 0.8789 | 31.46% |
| H8 | 0.43 | 0.6892 | 46.32% |
| H9 | 0.78 | 0.8869 | 12.83% |
| H10 | 0.47 | 0.5452 | 14.81% |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. (canceled)

2. (canceled)

3. (canceled)

4. A method for treating cancer in a cancer patient identified as having low [AAG] comprising a step of treating the patient with ARRY-520, comprising: (a) identifying the patient as having low [AAG] by assaying a biological sample from the patient, and (b) administering ARRY-520 to the patient having low [AAG].

5. (canceled)

6. A method of detecting a patient more likely to respond to ARRY-520, comprising obtaining a biological sample from the patient and assaying the sample to determine the [AAG], wherein low [AAG] is indicative of a patient more likely to respond to ARRY-520.

7. (canceled)

8. (canceled)

9. A method for increasing the likelihood of response in a patient having cancer, comprising: (a) obtaining a biological sample from the patient; (b) contacting the sample with an assay to measure the [AAG]; (c) determining whether the sample has low [AAG]; (d) classifying the patient as having an increased likelihood of response if the patient has low [AAG]; and (e) administering ARRY-520 to the patient classified as having an increased likelihood of response.

10. (canceled)

11. (canceled)

12. A method for determining a higher likelihood of sensitivity to ARRY-520 therapy in a cancer patient comprising: (a) assaying a biological sample from the patient for [AAG]; and (b) identifying the patient as having a higher likelihood of sensitivity to ARRY-520 therapy when the biological sample is low in [AAG].

13. (canceled)

14. A method of using ARRY-520 to treat a patient who has been diagnosed with levels of [AAG] of less than about 1.1 g/L, comprising administering one or more unit doses of ARRY-520.

15. (canceled)

16. A method of treating cancer in a patient having low [AAG], comprising administering to the patient an effective amount of ARRY-520.

17. (canceled)

18. (canceled)

19. (canceled)

20. (canceled)

21. (canceled)

22. The of claim 4, wherein the cancer is a hematological cancer.

23. The method of claim 22, wherein the cancer is selected from lymphomas, leukemia and multiple myeloma.

24. The method of claim 4, wherein the cancer is a solid tumor.

25. The method of claim 24, wherein the cancer is selected from skin, breast, brain, cervical carcinoma, and testicular cancer.

26. The method of claim 24, wherein the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.

27. The method of claim 4, wherein the low [AAG] is less than about 1.1 g/L.

28. The method of claim 27, wherein the [AAG] is measured by R&D Systems, Inc. Quantikine Human α1-Acid Glycoprotein Immunoassay.

29. The method of claim 4, wherein dexamethasone is administered in combination with ARRY-520.

30. The method of claim 4, wherein bortezomib is administered in combination with ARRY-520.

31. The method of claim 4, wherein carfilzomib is administered in combination with ARRY-520.

32. The method of claim 4, wherein pomalidomide is administered in combination with ARRY-520.

33. The method of claim 4, wherein G-CSF is administered in combination with ARRY-520.

34. The method of claim 29, wherein G-CSF is administered in combination with ARRY-520.

35. The method of claim 30, wherein G-CSF is administered in combination with ARRY-520.

36. The method of claim 31, wherein G-CSF is administered in combination with ARRY-520.

37. The method of claim 32, wherein G-CSF is administered in combination with ARRY-520.
